(19) United States
(12) Patent Application Publication
(10) Pub. No.: US 2024/0226159 A9
(43) Pub. Date: Jul. 11, 2024

(54) LOW INTENSITY ULTRASOUND COMBINATION CANCER THERAPIES

(71) Applicant: BOARD OF REGENTS, THE UNIVERSITY OF TEXAS SYSTEM, Austin, TX (US)

(72) Inventor: Amy B. HEIMBERGER, Houston, TX (US)

(73) Assignee: BOARD OF REGENTS, THE UNIVERSITY OF TEXAS SYSTEM, Austin, TX (US)

(21) Appl. No.: 18/546,141

(22) PCT Filed: Mar. 9, 2022
(86) PCT No.: PCT/US2022/019436

§ 371 Date: Aug. 11, 2023

Related U.S. Application Data
(60) Provisional application No. 63/158,642, filed on Mar. 9, 2021.

(51) Int. Cl. A61K 35/17; A61K 38/19; A61K 41/00; A61P 35/00; C07K 16/28

(52) U.S. Cl. CPC A61K 35/17; A61K 38/195; A61K 41/0033; A61P 35/00; C07K 16/2818; A61K 2039/505

(57) ABSTRACT

Provided herein are compositions, such as, for example, CXCL 10-secreting antigen presenting cells, and methods for ultrasound-induced blood-brain bander disruption (e.g., low-intensity pulsed ultrasound (LIPU)) to treat a brain cancer in a mammalian subject.

This application claims the benefit of U.S. Provisional Patent Application No. 63/158,642, filed Mar. 9, 2021, the entirety of which is incorporated herein by reference.

### BACKGROUND

### 1. Field

The present invention relates generally to the fields of medicine and oncology. More particularly, it concerns methods of treating a tumor patient with a low-intensity pulsed ultrasound (LIPU) therapy and/or antigen presenting cells modified to express a chemokine (e.g., CXCL10).

### 2. Description of Related Art

Currently there is a large compendium of immune therapeutics including immune checkpoint inhibitors, immune modulators, and cellular therapeutics that are hindered by the lack of blood brain barrier (BBB) penetration—either by direct interaction within the glioma microenvironment or through the paucity of immune effector trafficking. The BBB serves as a specific impediment to the delivery of large molecules and antibodies. Specifically, less than 1% of administered antibodies can usually be detected in the central nervous system (CNS); although they can be engineered to have increased CNS penetration (Ledford, 2011), this is unlikely to be feasible for many agents. Gliomas are immunologically unique in that they are enriched for some types of immune cells, such as macrophages (Wang et al., 2018; Wei et al., 2019), but are relatively lacking in others, such as T and NK cells (Hussain et al., 2006), which are capable of exerting direct tumor cytolytic activity. Even in the setting of active immunotherapy with agents such as immune checkpoint inhibitors, there may not be sufficient enrichment of effector T cells for tumor eradication. The T cell, in particular, can be sequestered in the bone marrow in patients with intracranial tumors (Chongsathidkiet et al., 2018). As such, unique strategies need to be considered for enhancing the presence of cellular therapeutics in tumors that reside in the CNS.

### SUMMARY

The present disclosure, in some aspects, provides compositions and methods for the treatment of cancer. In some embodiments, modified antigen presenting cells (e.g., modified to express CXCL10) and low-intensity pulsed ultrasound therapies are provided and may be used to treat a brain cancer, such as for example a glioma or glioblastoma.

An aspect of the present disclosure relates to a method of treating a brain cancer in a patient in need thereof, the method comprising administering to the patient an effective amount of antigen presenting cells (APCs) in conjunction with ultrasound blood brain barrier disruption, wherein the antigen presenting cells have been genetically modified to express a chemokine. The chemokine can be, e.g., CXCL9 or CXCL10. In some embodiments, the chemokine is CXCL10. In some embodiments, the administration is intravenous. The ultrasound blood brain barrier disruption may be a low-intensity pulsed ultrasound (LIPU) therapy (e.g., using an about 0.5-1.5, 0.9-1.2, 1-1.1, 0.6, 0.7, 0.8, 0.9, 1, 1.1, 1.2, 1.3, 1.4 or MPa pressure, or any range derivable therein, at about 0.2-10, 0.2-5, 0.2-0.4, 0.5-1.5, 0.2-0.5-2, 0.9-1.2, or 1-1.1 MHz, or any range derivable therein; or having a mechanical index of about 0.1-2, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, or any range derivable therein). The LIPU therapy may comprise ultrasound exposure in the presence of microbubbles or an ultrasound contrast agent (e.g., sulfur hexafluoride microbubbles, perflutren lipid microspheres, etc.). The microbubbles may be lipid-coated echogenic microbubbles. In some embodiments, the lipid-coated echogenic microbubbles are filled with octafluoropropane gas (e.g., Definity®). The microbubbles may be microspheres. In some embodiments, the microspheres are microspheres of human serum albumin with perflutren (e.g., OPTISON™). In some embodiments, the patient has previously failed to respond to an immunotherapy. The APC may be genetically modified to reduce or prevent immune suppression by the subject. The APC may be genetically modified to increase expression of MHC. The APC may be genetically modified to increase expression of one or more co-stimulatory molecules. In some embodiments, the APCs are derived from autologous cells from patient. In some embodiments, the APCs are derived from allogeneic cells. The APCs may be professional APCs, dendritic cells (DCs), macrophages, or B cells. The method may further comprises administering an immune checkpoint inhibitor to the subject. The immune checkpoint inhibitor may comprise an anti-PD1 antibody. The anti-PD1 antibody may comprise or consist of nivolumab, pembrolizumab, pidilizumab, AMP-223, AMP-514, cemiplimab, or PDR-001. The brain cancer may be a glioma, a glioblastoma, a glioblastoma multiforme, an astrocytoma, a pituitary adenoma, an acoustic neuroma, a medulloblastoma, a meningioma, a haemangioblastoma, an ependymoma, or a subependymoma. The method may further comprise administering a further anti-cancer therapy to the patient. The further anti-cancer therapy may be a chemotherapy, an immunotherapy, a radiotherapy, a gene therapy, surgery, a hormonal therapy, an anti-angiogenic therapy, or a cytokine therapy.

Another aspect of the present disclosure relates to an isolated, engineered antigen presenting cell (APC) that comprises a transgene for expressing at least one chemokine. In some embodiments, the chemokine is CXCL9 or CXCL10. In some preferred embodiments, the chemokine is CXCL10. The APC may be genetically modified to prevent its immune suppression. The APC may be genetically modified to fortify expression of MHC. In some embodiments, the APC is genetically modified to fortify expression of co-stimulatory molecules. In some embodiments, the APC is a professional APC, a macrophage, a dendritic cell, a T cell, or a B cell.

Yet another aspect of the present disclosure relates to a pharmaceutical composition comprising the APC described above or herein and at least one pharmaceutically acceptable carrier, diluent, or excipient. The composition may further comprise an immune checkpoint inhibitor. The pharmaceutical composition may be for use in the treatment of a brain cancer.

Another aspect of the present disclosure relates to a method of treating a brain cancer in a patient in need thereof, the method comprising administering to the patient an effective amount of the pharmaceutical composition described above or herein. In some embodiments, the administration is intracranial or intravenous. In some embodiments, the patient is subjected to ultrasound blood brain barrier disruption. In some preferred embodiments, the blood brain barrier disruption is low-intensity pulsed ultrasound (LIPU) therapy (e.g., using an about 0.5-1.5, 0.9-1.2, 1-1.1, 0.6, 0.7, 0.8, 0.9, 1, 1.1, 1.2, 1.3, 1.4 or MPa pressure, or any range derivable therein, at about 0.2-10, 0.2-5, 0.2-0.4, 0.5-1.5, 0.5-2, 0.9-1.2, or 1-1.1 MHz, or any range derivable therein; or having a mechanical index of about 0.1-2, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, or any range derivable therein). The LIPU therapy may comprise ultrasound exposure in the presence of microbubbles or an ultrasound contrast agent (e.g., sulfur hexafluoride microbubbles, perflutren lipid microspheres, etc.). The microbubbles may be lipid-coated echogenic microbubbles. In some embodiments, the lipid-coated echogenic microbubbles are filled with octafluoropropane gas (e.g., Definity®). In some embodiments, the microbubbles are microspheres. The microspheres may be microspheres of human serum albumin with perflutren (e.g., OPTISON™). In some embodiments, the patient has previously failed to response to an immunotherapy. The method may reduce or prevent tumor recurrence. The APCs may be autologous to the patient. In some embodiments, the APCs are allogeneic to the patient. The brain cancer may be, e.g., a glioma, a glioblastoma, a glioblastoma multiforme, an astrocytoma, a pituitary adenoma, an acoustic neuroma, a medulloblastoma, a meningioma, a haemangioblastoma, an ependymoma, or a subependymoma. The method may further comprise administering a further therapy to the patient. The further therapy may be a chemotherapy, an immunotherapy, a radiotherapy, a gene therapy, surgery, a hormonal therapy, an anti-angiogenic therapy, or a cytokine therapy. The immunotherapy may comprise an immune checkpoint inhibitor. The immune checkpoint inhibitor may comprise or consist of an anti-PD1 antibody (e.g., nivolumab, pembrolizumab, pidilizumab, AMP-223, AMP-514, cemiplimab, or PDR-001). The immunotherapy may comprise an adoptive T-cell therapy.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-1D. Ultrasound mediated blood brain barrier disruption (BBBD) causes reproducible and targeted BBB opening. (FIG. 1A) Left panel: Pre-clinical platform for BBB opening in murine models. Right panel: The mouse calvaria is shaved and the mouse is positioned supine. The region for BBBD is positioned directly over the ultrasound pulse area using laser guidance (red mark) after the head is secured using the elastic bands. (FIG. 1B) Schema of the treatment of non-tumor bearing GL261 mice with intravenous (i.v.) Evans blue dye followed by immediate i.v. microbubble injection and ultrasound administration. Mouse perfusion was followed by gross brain analysis done 45 minutes after Evans blue injection. (FIG. 1C) Representative photographs of whole brains from superior and inferior projections taken immediately after mouse perfusion and brain dissection. (FIG. 1D) Perfused whole brains as shown in FIG. 1C that were coronally sectioned from anterior to posterior in the same order from left to right as FIG. 1C. These figures demonstrate that the preclinical BBBD device is adequately opening the BBB as demonstrated by the staining of Evans Blue.

FIGS. 2A-2F. Anti-PD-1 administered with ultrasound BBBD causes increased brain delivery of the antibody and enhanced murine survival. (FIG. 2A) Treatment schema of labeled anti-PD-1 administered with ultrasound to non-tumor bearing GL261 mice. Labeled antibody circulated in mice for 3 hours prior to perfusion and brain dissection. (FIG. 2B) Fluorescent imaging was performed using IVIS Spectrum on whole and coronally sectioned brains immediately after mouse perfusion and brain dissection following the treatment described in FIG. 2A. Mice were treated with labeled anti-PD-1 antibody alone (left column), labeled anti-PD-1 and one sonication (middle column), or labeled anti-PD-1 and two sonications (right column). (FIG. 2C) Treatment schema of immune competent mice that underwent i.c. implantation of GL261 cells followed by treatment with anti-PD-1. Three days after GL261 implantation, anti-PD-1 was administered two times per week for a total of 5 treatments. (FIG. 2D) Survival of C57BL/6 mice treated with IgG control, anti-PD-1, or anti-PD-1 with ultrasound. Survival analysis of the mice was performed using the log-rank (Mantel-Cox) test. IgG: 6 mice [median survival (MS): 22 days], anti-PD-1: 6 mice [39 days], anti-PD-1+ultrasound: 6 mice [>58 days]. Statistics: IgG vs anti-PD-1 p=0.007; IgG vs anti-PD-l+US p=0.0022; anti-PD-1 vs anti-PD-1+US p=0.6226. This experiment was repeated in its entirety with similar findings. (FIG. 2E) Treatment schema of long-term survivors that were re-challenged with the same concentration of GL261 cells in the contralateral hemisphere. Naive controls were age matched to the long-term survivors. (FIG. 2F) Kaplan-Meier survival of long-term survivors from FIG. 2E re-challenged in the contralateral hemisphere and naive age-matched mice challenged in the same hemisphere. Naive control: 10 mice [MS: 21 days], anti-PD1: 2 mice [MS: undefined], anti-PD-1+ultrasound: 3 mice [MS: undefined]. Statistics: Naive control vs anti-PD-1 p=0.0148: Naive control vs anti-PD-1+ultrasound p=0.0044. These figures illustrate that BBBD can enhance the delivery of immunotherapeutic antibodies to CNS tumors.

FIGS. 3A-3D. I.V. administered chimeric antigen receptor (CAR) T cells in combination with ultrasound BBBD increases trafficking to the brain and persistence in the tumor microenvironment. (FIG. 3A) Treatment schema for BLI of ffLuc CAR T cells administered i.v. in EGFRvIII-U87 tumor implanted mice. BLI was performed at 5, 24, and 72 hours and at 7 days after CAR T cell treatment. (FIG. 3B) Expression of the EGFRvIII CAR was verified by flow cytometry using anti-Fc antibody for the IgG4 extracellular domain of the CAR prior to administration. The infused EGFRvIII CAR product contained both CD4 and CD8 T cell populations as demonstrated by flow cytometry in the lymphocyte population. (FIG. 3C) Representative example from BLI of ffLuc from 1.5×10⁷ EGFRvIII/ffLuc CAR T cells administered i.v., without ultrasound (left) or with ultrasound (right), in EGFRvIII-U87 tumor implanted mice. Red box demonstrates BLI measurement from CAR T cells present in the head region. (FIG. 3D) Summary of BLI signal from head regions in EGFRvIII/ffLuc treated mice at 5 hours, 24 hours, 72 hours, and 7 days. Statistics: 24 hours non-ultrasound vs. ultrasound p=0.0043, 72 hours non-ultrasound vs. ultrasound p<0.0001. Statistical analysis was performed using the F test to compare variances. 5 hr: non-US (n=5), US (n=9); 24 hr: non-US (n=5), US (n=9); 72 hr: non-US (n=4), US (n=9); 7 days: non-US (n=3), US (n=9). These figures illustrate that BBBD can enhance the delivery of CAR T cells to CNS tumors.

FIGS. 4A-4B. Ultrasound mediated BBBD in combination with CAR T cell therapy is associated with an increase in murine survival. (FIG. 4A) Treatment schema of NSG mice that underwent i.c. implantation of 150,000 EGFRvIII-U87 cells and were treated on day 14 with i.v. 1.5×10⁷ EGFRvIII/ffLuc CAR T cells. (FIG. 4B) Survival of NSG mice treated with EGFRvIII CAR T cell, or EGFRvIII CAR T cell with ultrasound. Survival was determined using Kaplan-Meier survival analysis with one-tailed t test for independent samples. EGFRvIII CAR T cell: 4 mice [MS: 35 days], EGFRvIII CAR T cell+ultrasound: 7 mice [MS: undefined]. Statistics: EGFRvIII CAR T-cell vs EGFRvIII CAR T cell+ultrasound p=0.04423. These figures illustrate that BBBD can enhance the therapeutic effect of CAR T cells to CNS tumors when administered systemically.

FIGS. 5A-5E. Antigen presenting cells (APCs) expressing CXCL10 administered i.v. with US-induced BBBD are associated with a significant increase in immune competent murine survival. (FIG. 5A) Schema of the lentivirus gene transfer plasmid structure for either the murine CXCL9 or CXCL10. (FIG. 5B) Schema of the gene transfer plasmid of green fluorescent protein (GFP) alone to serve as the control. (FIG. 5C) Treatment schema of C57BL/6 that received i.c. implantation of 50,000 GL261 cells and were treated on day 7 with 1×10⁶ CXCL9 or CXCL10 APCs. (FIG. 5D) Survival of immune competent mice treated with PBS, i.c. CXCL9 APCs, i.v. CXCL9 APCs, or i.v. CXCL9 APCs with ultrasound. Survival analysis was performed using the log-rank (Mantel-Cox) test. PBS: 3 mice [MS: 24 days], i.c. CXCL9 APCs: 4 mice [MS: 24], i.v. CXCL9 APCs: 4 mice [MS: 22], i.v. CXCL9 APCs+ultrasound: 4 mice [MS: 25.5 days]. Statistics: PBS vs i.c. CXCL9 APCs p=0.7764; PBS vs i.v. CXCL9 APCs p=0.9298; PBS vs i.v. CXCL9 APCs+ultrasound p=0.4805; i.c. CXCL9 APCs vs i.v. CXCL9 APCs p=0.6124: i.c. CXCL9 APCs vs i.v. CXCL9 APCs+ultrasound p=0.1962; i.v. CXCL9 APCs vs i.v. CXCL9 APCs+ultrasound p=0.4524. (FIG. 5E) Survival of immune competent mice treated with PBS, i.c. CXCL10 APCs, i.v. CXCL10 APCs, or i.v. CXCL 10 APCs+ultrasound. PBS: 3 mice [MS: 24 days], i.c. CXCL10 APCs: 4 mice [MS: 28 days], i.v. CXCL10 APCs: 4 mice [MS: 24 days], i.v. CXCL10 APCs+ultrasound: 3 mice [MS: 34 days]. Statistics: PBS vs i.c. CXCL10 APCs p=0.0476; PBS vs i.v. CXCL10 APCs p=0.6041; PBS vs i.v. CXCL10 APCs+ultrasound p=0.0246; i.c. CXCL10 APCs vs i.v. CXCL10 APCs p=0.6349; i.c. CXCL10 APCs vs i.v. CXCL10 APCs+ultrasound p=0.0213; i.v. CXCL10 APCs vs i.v. CXCL10 APCs+ultrasound p=0.0415. This figure demonstrates that BBBD deposition of genetically modified APC into the CNS tumor induces a therapeutic response.

FIGS. 6A-6C. Schemas demonstrating how ultrasound mediated BBBD delivers antibodies, CAR T cells, and APCs to the tumor microenvironment for immune activation and improved tumor cell killing. (FIG. 6A) Anti-PD-1 delivered through an open BBB blocks immune exhaustion on effector T cells thereby licensing the T cell to exert an effector response through perforin and/or granzyme B. (FIG. 6B) Ultrasound administered CAR T cells are able to more diffusely infiltrate the tumor microenvironment with longer persistence, thereby triggering tumor cytotoxicity through the chimeric antigen receptor (CAR). (FIG. 6C) Ultrasound BBB opening allows APCs and T cells to infiltrate the tumor microenvironment. These APCs have been genetically modified to secrete the T cell cytokine CXCL10. Secondary to the APCs presenting antigens to the T cell, the T cell becomes activated and thereby can mediate direct tumor killing since the T cells have not been chronically stimulated.

### DETAILED DESCRIPTION

Ultrasound (US)—induced opening of the BBB is a promising technique that could facilitate the entry of a wide range of substances, such as antibodies and cells into the glioma microenvironment (Alkins et al., 2013; Hynynen et al., 2001; Kinoshita et al., 2006). In the context of immunotherapy, this strategy could be used to increase the concentrations of antibodies whose target resides in the CNS. Such targets could either be tumor antigens or immune modulatory antibodies in which the target cell population, such as microglia, resides in the CNS. In addition, US-induced opening of the BBB enhances the therapeutic impact in preclinical models of a wide variety of immune therapeutics that have either been tested in glioblastoma patients and failed or a novel cellular strategy that demonstrated no evidence of efficacy previously. These studies show that the use of ultrasound induced BBBD can enhance the therapeutic effects of a variety of immune therapeutic strategies for gliomas by enhancing delivery of antibodies, chimeric antigen receptor T cells, and genetically modified cellular immune therapeutics such as an antigen presenting cell to the tumor microenvironment that can be rapidly translated to clinical trials.

As shown in the examples, data is provided showing enhancement of immunotherapies and cell therapies by US, and in particular low-intensity pulsed ultrasound (LIPU), to open the BBB. These results are contrary to conventional theory. Conventional theory holds that antigen presentation and immune activation are triggered in the peripheral lymphatics. The activated T cell subsequently exits and travels to the tumor microenvironment, guided by a gradient of tumor-elaborated chemokines, to exert tumor destruction. As such, the deposition of an antigen presenting cell in the tumor microenvironment by BBBD is contrary to this prevailing theory. Without wishing to be bound by any theory, because professional APC such as dendritic cells are very rare gliomas but are abundant in brain metastasis (Nwajei et al., 2015), it is anticipated that deposition of APC in the tumor microenvironment may induce T cell migration into the tumor with subsequent T cell activation.

### I. ASPECTS OF THE PRESENT DISCLOSURE

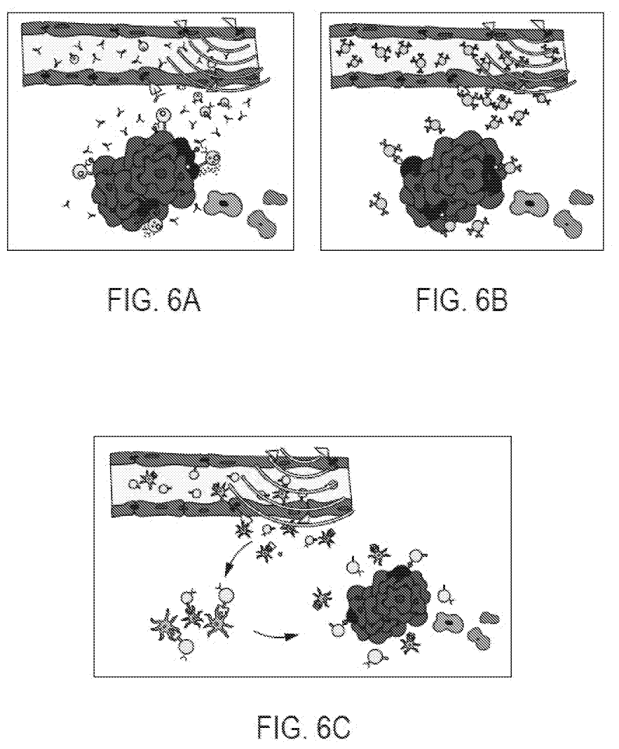

There are certain immune cells that are desirable to increase in the glioma microenvironment with US-induced BBBD, such as CD8+ cytotoxic T cells (e.g., adoptive T cells, CARs, etc.) and cytotoxic NK cells, because there are so few of these effector cells in the tumor microenvironment. However, this needs to be specific enough not to also enhance undesirable tumor supportive and immune suppressive cells such as Tregs, M2 macrophages, and myeloid derived suppressors. As such, the present disclosure provides for the delivery of specific immune cell subtypes and immune modulators that would favorably tip the balance toward pro-inflammatory responses as opposed to just a generalized increase in immune cells overall. The most immediate and more globally available strategy tested that could be implemented in a clinical trial is the use of anti-PD-1. Ultrasound was not only able to better deliver anti-PD-1, but also provided a significant improvement in survival, even when a tumor recurrence was mimicked using contralateral hemisphere re-challenge. Previous studies have shown immune checkpoint inhibitor mediated immune memory responses (Belcaid et al., 2014; Reardon et al., 2016; Ribas et al., 2016; Vom Berg et al., 2013). By enhancing both T cell and anti-PD-1 delivery across the BBB with ultrasound, additional CD4 and CD8 cell dysfunction in the tumor microenvironment may be prevented, thereby allowing for greater effector responses (FIG. 6A). BBB opening ultrasound also enhanced the in vivo persistence in the tumor microenvironment of CAR T cells that correlated with increased survival. Improved CAR T cell trafficking and persistence with ultrasound allows CAR mediated recognition of tumor antigens for increased tumor cell killing (FIG. 6B). The delivery of the CXCL10-secreting antigen presenting cells to the glioma microenvironment with US-induced BBBD was even superior to direct intracranial injection. Without being bound by any theory, this may have been because of more diffuse dispersal of the APCs throughout the tumor microenvironment relative to the direct injection which may have been more focal. Alternatively, the passage of the APC through the perivascular region after US-induced BBBD may have positioned APCs in closer proximity to the T cells as they immigrate from the vascular space into the localized glioma microenvironment. Overall, the data provided in this disclosure indicates that this strategy can enhance the therapeutic index of a wide variety of immune therapy strategies for patients with CNS tumors.

Current dogma is that anti-glioma immune responses are generated from the lymphatic drainage of antigens to the cervical lymph nodes (Louveau el al., 2015) where professional APCs like dendritic cells present them to T cells, which then trigger their cytotoxic effector functions (Mitchell et al., 2015). These T cells then traffic to the local tumor microenvironment to eradicate the glioma. However, immune phenotyping reveals these T cells lack effector function and are exhausted (Woroniecka et al., 2018). Chronic T cell stimulation with weak tumor antigens will precipitate this state (Wherry, 2011). The TCR repertoire is different between the lymph node and the primary cancer (Wang et al., 2017b) and even distinct within various regions of the malignancy itself (Reuben et al., 2017) indicating differences in antigen profiles in various anatomical locations. To date, matched antigenic profiling between the tumor and lymph node has not been conducted, but based on the TCR repertoire analysis, it would be plausible that there are antigenic differences. During the initial antigen presentation event, immune checkpoints are not yet significantly up regulated (Sabins et al., 2016) and are not exhausted, thus, T cells are free to exert their effector responses including eradication of the tumor. If there were sufficient APCs in the glioma microenvironment capable of presenting novel antigens to a naïve T cell, then these cells could be capable of inducing anti-tumor effector responses. Based on nanostring profiling between primary gliomas and brain metastasis, marked differences were found in the frequency of activated dendritic cells in the CNS tumor microenvironment with activated dendritic cells (a type of APCs) being almost completely absent in high grade gliomas. Furthermore, marked dendritic cell and T cell cluster interactions can be induced in preclinical models of gliomas using a combination of radiation and maintenance of dendritic cell activation with a STAT3 inhibitor (Ott et al., 2019; Nefedova et al., 2005). As such, the use of US-induced BBBD to deposit APCs into the tumor microenvironment in a uniform and consistent manner was evaluated. Ultrasound mediated deposition of CXCL10-expressing APCs loads the tumor microenvironment with T cells that become activated and directed for killing of tumor cells (FIG. 6C). Bone marrow derived APCs were stably transfected with either the T cell chemokines CXCL9 or CXCL10 and used to treat mice with established gliomas. APCs secreting CXCL10 induced a significant increase in survival.

### I. DEFINITIONS

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The terms “subject,” “host,” “patient,” and “individual” are used interchangeably herein to refer to any mammalian subject for whom therapy is desired, particularly humans. Other subjects may include cattle, dogs, cats, guinea pigs, rabbits, rats, mice, horses, and so on.

The term “unit dose” when used in reference to a therapeutic composition refers to physically discrete units suitable as unitary dosage for the subject, each unit containing a predetermined quantity of active material calculated to produce the desired therapeutic effect in association with the required diluent, i.e., carrier, or vehicle.

The terms “cell,” and “cells,” and “cell population,” used interchangeably, intend one or more mammalian cells. The term includes progeny of a cell or cell population. Those skilled in the art will recognize that “cells” include progeny of a single cell, and there are variations between the progeny and its original parent cell due to natural, accidental, or deliberate mutation or change.

The term “immunotherapy” refers to treatment of disease (e.g., cancer) by modulating an immune response to a disease antigen.

The term “cancer cell” as used herein refers to a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth or development in an immunocompromised non-human animal model, or any appropriate indicator of cellular transformation. “Cancer cell” may be used interchangeably herein with “tumor cell” or “cancerous cell” and encompasses cancer cells of a solid tumor and a liquid tumor. “Cancer” may be used interchangeably herein with “tumor”.

The term “effective amount” is an amount sufficient to effect beneficial or desired clinical results. An effective amount can be administered in one or more administrations. For purposes of this application, an effective amount is an amount that is sufficient to palliate, ameliorate, stabilize, reverse, slow or delay the progression of the disease state. This includes, but is not limited to, a reduction in the frequency or severity of the signs or symptoms of a disease. For example, treatment of cancer may involve, for example, a reduction in the invasiveness of a tumor, reduction in the growth rate of the cancer, or prevention of metastasis. Treatment of cancer may also refer to prolonging survival of a subject with cancer.

An effective response of a patient or a patient's “responsiveness” to treatment refers to the clinical or therapeutic benefit imparted to a patient at risk for, or suffering from, a disease or disorder. Such benefit may include cellular or biological responses, a complete response, a partial response, a stable disease (without progression or relapse), or a response with a later relapse. For example, an effective response can be reduced tumor size or progression-free survival in a patient diagnosed with cancer.

The terms “in operable combination”, “in operable order”, and “operably linked” refer to a linkage wherein the components so described are in a relationship permitting them to function in their intended manner, for example, a linkage of nucleic acid sequences in such a manner that a nucleic acid molecule capable of directing the transcription of a given gene or the synthesis of desired protein molecule, or a linkage of amino acid sequences in such a manner so that a fusion protein is produced.

“Treatment” and “treating” refer to administration or application of a therapeutic agent to a subject or performance of a procedure or modality on a subject for the purpose of obtaining a therapeutic benefit of a disease or health-related condition.

### III. ULTRASOUND OPENING OF THE BLOOD-BRAIN BARRIER (BBB)

Low intensity pulsed ultrasound (LIPU) in combination with gaseous microbubbles has emerged as a potential new means of effective drug delivery to the brain. Ultrasound energy can be delivered to the brain using either implantable ultrasound devices, transcranial focused ultrasound devices, or through acoustic windows implanted in the skull bone. Recent research has shown that, under low duty cycle pulsing type energy exposure with the presence of microbubbles, this modality can transiently permeate the blood-brain barrier (BBB). The bioavailability of therapeutic agents is site-specifically augmented only in the zone where the ultrasound energy is targeted. This strategy can enhance CNS penetration of therapeutic agents including chemotherapeutic agents, therapeutic peptides, monoclonal antibodies, and nanoparticles. In some preferred embodiments, the ultrasound is low-intensity pulsed ultrasound (LIPU).

The blood-brain barrier (BBB) serves as a key homeostatic site for the central nervous system (CNS) and is involved with maintaining structural and functional brain connectivity (Zhao et al., 2015). The BBB is composed of specialized highly polarized endothelial cells, pericytes, and astrocytic processes. The capillary endothelium composes the majority of the BBB surface area (>85%) and a variety of transport systems facilitate or actively shuttle molecules across the BBB (Sweeney et al., 2018). Dysfunction of BBB permeability and transporters lead to various kinds of neurological disorders, including stroke, Alzheimer's, Huntington's, Parkinson's, amyotrophic lateral sclerosis, multiple sclerosis, various types of infectious disease and even neoplasms, which may alter the regional or even global cerebral microenvironment (Schoknecht et al., 2015; Nelson et al., 2016; Maiuolo et al., 2018; Sweeney et al., 2018). Therapeutic targets have been proposed to treat a broad spectrum of disease, but must first cross the BBB for effective drug delivery or to increase waste elimination (e.g., amyloid β) (Nelson et al., 2016; Sweeney et al., 2018).

The BBB blocks nearly 98% of drug compounds from accessing the CNS, and the use of LIPU raises the potential for developing a drug delivery platform (Pardridge, 2005). The permeability of the BBB can be transiently increased using low-energy burst-tone ultrasound following an administration of intravenous microbubbles (Hynynen et al., 2001, 2003; Park el al., 2012; Chai et al., 2014). A physical cavitation effect is created from circulating microbubbles, significantly reducing the ultrasound pressure to produce an equivalent acoustic cavitation effect. The subsequent application of ultrasonic energy can achieve a local detachment of tightly sealed junctions on the capillary wall without inducing neuronal damage (Hynynen et al., 2005). Since the BBB blocks nearly 98% of drugs from accessing the CNS, the use of pulsed ultrasound raises a potential therapeutic delivery platform to the CNS (Pardridge, 2005).

Microbubbles (MB) can promote a FUS-induced BBB opening effect. MB are available from a variety of commercial sources, including Optison (GE Healthcare, WI, USA), Definity® (Lantheus Medical Imaging, MA, USA), and SonoVue® (Bracco, Milano, Italy). These MBs received FDA approval for diagnostic use and have been used for ultrasound-induced BBB opening. Commercial MBs typically are about 1-10 μm in diameter and may have an application window of about 5-10 min. MBs can vary in their composition, concentrations, half-lives, and/or hydrodynamic sizes. Intravenously injected microbubbles administered during the delivery of low intensity pulsed ultrasound can induce transient opening of the BBB. The mechanisms underlying US-induced BBBD are not totally elucidated but may be based on the expansion and contraction of injected microbubbles, also called cavitation, and different cellular mechanisms may be involved, including: (1) transcytosis, (2) transendothelial openings (fenestrations, channels formation), (3) opening of the tight junctions and interendothelial clefts, and/or (4) free passage of molecules through the permeable endothelium at higher ultrasonic energies.

A variety of devices can be used in the ultrasound methods as disclosed herein (e.g., LIPU with gaseous microbubbles). In some embodiments, the SonoCloud® device is used to achieve temporary disruption of the BBB. The SonoCloud® is an implantable medical device that is fixed to the skull and does not contain any integrated power source. The upper face, which is in contact with the underside of the skin, contains a connection channel that receives a bipolar needle which is introduced through the skin at each use. The bottom face, which is in contact with the dura mater and brain, consists of a transducer that emits ultrasound energy to open the blood-brain barrier. The SonoCloud® device is powered and controlled by an external generator that verifies the quality of the electrical connection, delivers radio-frequency energy to power the device, and saves the entire procedure sequence. The SonoCloud® device is described, e.g., in (Carpentier e al., 2016).

Devices that can be used to apply ultrasound as described herein to the BBB include SonoCloud® (CarThera), ExAblate® (InSightec), and NaviFUS® (NaviFUS cooperation), and the devices can be used with or without chemotherapy regimens (e.g., carboplatin, doxorubicin, and/or temozolomide). A repeated opening of the BBB using a 1 MHz implanted pulsed ultrasound device (SonoCloud®), in combination with Sonovue® (dose: 0.1 ml/kg) at an acoustic pressure ranged from 0.5 to 1.1 MPa, has been shown to be safe and well tolerated in treating recurrent GBM patients (Carpentier et al., 2016).

LIPU with microbubbles is used in some preferred embodiments disclosed herein to promote opening of the BBB. High-intensity focused-ultrasound (HIFU) has been successfully employed for thermal ablation of tumors in clinical settings. Continuous- or pulsed-mode HIFU may also induce a host antitumor immune response, e.g., through expansion of antigen-presenting cells in response to increased cellular debris and through increased macrophage activation/infiltration. In contrast to HIFU, LIPU uses focused ultrasound delivery with low-pressure, pulsed-mode exposure in the presence of microbubbles (MBs) or an ultrasound contrast agent. LIPU can also trigger an antitumor immunological response and inhibit tumor growth.

A variety of ranges of lower pressure can be used to promote opening of the BBB. For example, about 0.5-1.5, 0.9-1.2, 1-1.1, 0.6, 0.7, 0.8, 0.9, 1, 1.1, 1.2, 1.3, 1.4 or MPa pressures, or any range derivable therein, can be applied with MBs in a LIPU therapy in the methods disclosed herein; the pressure may be applied for example at about 0.2-10, 0.2-5, 0.2-0.4, 0.5-1.5, 0.5-2, 0.9-1.2, or 1-1.1 MHz. In some embodiments, the LIPU is applied at about 0.2-0.4 MHz with transcranial stimulation. Nonetheless, BBB opening can be achieved at higher frequencies, including as high as about 10 MHz. In some embodiments the LIPU is applied at a frequency of 0.5-5 or 0.2-10 MHz. For example, in some embodiments 1.03 MPa at 1 MHz is used in a LIPU therapy. The mechanical index (MI), which is the negative acoustic pressure (P- in MPa) divided by the square root of the frequency in MHz, can also be used to measure the pressure applied to promote opening of the BBB. In some embodiments, the MI is about 0.1-2, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, or any range derivable therein. In some embodiments, the ultrasound contrast agent or MBs that are used with the LIPU are perflutren lipid microspheres, lipid-coated echogenic microbubbles (e.g., lipid-coated echogenic microbubbles filled with octafluoropropane gas), microspheres of human serum albumin with perflutren, or sulfur hexafluoride microbubbles. A variety of ultrasound contrast agents are commercially available that may be used in various embodiments including, e.g., Definity®, SonoVue®, and Optison®. It is anticipated that BBB opening and effects described herein can be achieved with essentially any type of microbubble or ultrasound contrast agent (e.g., Definity®, SonoVue®, Optison®, etc.). In some embodiments, the ultrasound contrast agent is a perflutren lipid microsphere. Definity® is one example of a commercially available perflutren lipid microspheres filled with octafluoropropane gas that can be used in some embodiments of the present disclosure. In some embodiments, the MBs are lipid-coated echogenic microbubbles, such as for example lipid-coated echogenic microbubbles filled with octafluoropropane gas. In some embodiments, focused ultrasound methodologies described in Song et al. (2017) can be used to promote BBB opening in various methods of the present disclosure.

In some embodiments, the following methods are used for LIPU opening of the BBB. Patients with a brain tumor (e.g., GBM or recurrent GBM) can be implanted with an ultrasound device (e.g., a 1 MHz, 10-mm diameter pulsed ultrasound device in a burr hole during additional debulking surgery or during a dedicated procedure under local anesthesia). Ultrasound dose can be escalated, e.g., using a 3+3 Simon design. The ultrasound device can be activated (e.g., monthly) in combination with injection of a microbubble (e.g., sulfur hexafluoride microbubble, lipid-coated echogenic microbubble, lipid-coated echogenic microbubble filled with octafluoropropane gas, perflutren lipid microspheres) to transiently disrupt the BBB in 5 cm³ of the tumor field. The median number of monthly sonications per patient can vary as desired, and may for example include 1-6, 2-5, 2-4, or 1, 2, 3, 4, 5, 6, 7, 8 9, or 10 sonications, or any range derivable therein. The disruption can be performed before or after administration of an anticancer therapy (e.g., after administration of modified antigen presenting cells as described herein, and/or before administration of a chemotherapeutic such as for example carboplatin). Patients may receive about 150-270 seconds of pulsed ultrasound. In some embodiments, the puled ultrasound is administered less than about 1 hour prior to or after administration of a drug therapy (e.g., a chemotherapy). BBB disruption can be visualized using contrast-enhanced T1w MRI if desired, and patients may be monitored clinically (e.g., with T2, FLAIR, DWI and SWI sequences). Tumor progression can be evaluated using the RANO criteria, if desired. In some embodiments, the patient has a GBM or glioma brain cancer.

### IV. ENGINEERED ANTIGEN PRESENTING CELLS

In certain embodiments, there are provided engineered APCs. Such cells may be administered to a patient and thereby stimulate expansion of immune effector cells in vivo, including within the CNS tumor microenvironment. As used herein the term “engineered APC” refers to a cell that comprises at least a first introduced gene encoding a chemokine. In some aspects, the chemokine may be CXCL10. The amino acid sequence and the cDNA sequence of human CXCL10, also called C7, IFI10, INP10, IP-10, SCYB10, crg-2, gIP-10, mob-1, C-X-C motif chemokine ligand 10, and C-X-C motif chemokine 10, are described in Genbank Accession Nos. NP_001556.2 (Protein) and NM_001565.4 (mRNA), each of which is incorporated herein by reference. In some aspects, the chemokine may be CXCL9. The amino acid sequence and the cDNA sequence of human CXCL9, also called CMK, Humig, MIG, SCYB9, crg-10, and C-X-C motif chemokine ligand 9, are described in Genbank Accession Nos. NP_002407.1 (Protein) and NM_002416.3 (mRNA), each of which is incorporated herein by reference.

In some aspects, an engineered APC of the embodiments may further comprise a gene encoding a co-stimulatory molecule. The co-stimulatory molecule may be a co-stimulatory cytokine that may be a membrane-bound Cγ cytokine. In certain aspects, the co-stimulatory cytokine is IL-15, such as membrane-bound IL-15. In some further aspects, an engineered APC may comprise an edited (or deleted) gene. For example, an inhibitory gene, such as PD-1, LIM-3, CTLA-4, can be edited to reduce or eliminate expression of the gene.

Exemplary types of APCs that may be used in the present disclosure include macrophages, dendritic cells, B cells, and T cells. Methods for isolating and culturing such cells are well known in the art. The APCs may be isolated from subjects, particularly human subjects. The APCs can be obtained from a subject of interest, such as a subject suspected of having a particular disease or condition, a subject suspected of having a predisposition to a particular disease or condition, a subject who is undergoing therapy for a particular disease or condition, a subject who is a healthy volunteer or healthy donor, or from a blood bank. APCs can be collected, enriched, and/or purified from any tissue or organ in which they reside in the subject including, but not limited to, blood, cord blood, spleen, thymus, lymph nodes, bone marrow, tissues removed and/or exposed during surgical procedures, and tissues obtained via biopsy procedures. The isolated immune effector cells may be used directly, or they can be stored for a period of time, such as by freezing.

A pharmaceutical composition of the embodiments can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the embodiments, alone or in combination with other active agents, calculated in an amount sufficient to produce the desired effect, in association with a pharmaceutically acceptable diluent, carrier, or vehicle, where appropriate. The specifications for the novel unit dosage forms of the embodiments depend on the particular pharmacodynamics associated with the pharmaceutical composition in the particular subject.

Desirably an effective amount or sufficient number of the engineered APCs is present in the composition, such that when introduced into the subject, long-term, anti-tumor responses are established to reduce the size of a tumor or eliminate tumor growth or regrowth than would otherwise result in the absence of such treatment. Desirably, the amount of engineered APCs administered to the subject causes a 10%, 20%, 30%, 40%, 50%, 60%, 70%, 80%, 90%, 95%, 98%, or 100% decrease in tumor size when compared to otherwise same conditions wherein the engineered APCs are not present.

Furthermore, the amounts of each active agent included in the compositions described herein can vary in different applications. In general, the concentration of engineered APCs desirably should be sufficient to provide in the subject being treated at least from about 1×10⁵ to about 5×10¹² engineered APCs, even more desirably, from about 1×10⁶ to about 1×10¹⁰ engineered APCs, although any suitable amount can be utilized either above, e.g., greater than 5×10¹² cells, or below, e.g., less than 1×10⁵ cells. As another example, suitable doses for a therapeutic effect would be at least 10⁵ or between about 10⁵ and about 10¹⁰ cells per dose, for example, optionally in a series of dosing cycles. An exemplary dosing regimen consists of four one-week dosing cycles of optionally escalating doses, starting at least at about 10⁵ cells on Day 0, for example increasing incrementally up to a target dose of about 10¹⁰ cells within several weeks of initiating an intra-patient dose escalation scheme.

These values provide general guidance of the range of engineered APCs to be used by the practitioner upon optimizing the method of the embodiments. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

### V. CANCERS AND METHODS OF TREATMENT

The present disclosure provides methods of treating a tumor patient with an ultrasound therapy (e.g., LIPU therapy) and/or antigen presenting cells modified to express a chemokine (e.g., CXCL10). Such treatment may optionally comprise administering an additional therapeutic regime, such as chemotherapy, immunotherapy, radiotherapy, performance of surgery, or any combination thereof. In some preferred embodiments the tumor is a brain tumor, the LIPU is applied to disrupt the BBB in the subject. It is nonetheless anticipated that the ultrasound therapy may comprise or consist of focused ultrasound application to a tumor in the subject outside of the brain.

In some embodiments, the tumor is a brain tumor. It is anticipated that a variety of brain tumors may be treated with the methods and compositions as disclosed herein. For example, the brain tumor may be a glioma, a glioblastoma or glioblastoma multiforme, a high grade intrinsic brain tumor, an astrocytoma (e.g., Grade I—pilocytic astrocytoma, Grade II—low-grade astrocytoma, Grade III—anaplastic astrocytoma, or Grade IV—glioblastoma (GBM)), a pituitary adenoma, an acoustic neuroma, a medulloblastoma, a meningioma, an oligodendroglioma, a haemangioblastoma, a CNS lymphoma, an unspecified glioma, a brain stem glioma, an ependymoma, a mixed glioma, an optic nerve glioma, a subependymoma, a brain stem glioma, a craniopharyngioma, an ependymoma, a juvenile pilocytic astrocytoma (JPA), a medulloblastoma, an optic nerve glioma, a pineal tumor, a primitive neuroectodermal tumor (PNET), a rhabdoid tumor, or a metastatic brain tumor.

GBM is a grade 4 astrocytoma. It is an aggressive cancer that grows from the supportive cells in the brain and is diffusely infiltrative. The current standard treatment is aggressive surgical debulking followed by combined modality therapy of chemotherapy and radiation. Despite the neurosurgeon successfully resecting all visible abnormal tissue during surgery, there are normally many cancer cells that extend well past the resection cavity and are still present in the patient after the surgery. The average survival rate for patients with glioblastoma multiforme who have had aggressive treatments, including surgical resection, radiotherapy and chemotherapy, has been reported to be about fourteen months. It has also been reported that less than 30% of patients survive two years. Long-term survival is extremely rare.

GBM has a complex growth pattern. There is typically a tumor mass (or more than one) that is easily detected with conventional imaging. The tumor mass does not have a sharp border. Instead, individual tumor cells infiltrate the brain parenchyma and may be widely disseminated at the time of diagnosis.

In certain embodiments, the compositions and methods of the present embodiments involve administration of a LIPU therapy and/or APCs modified to express a chemokine (e.g., CXCL10) in combination with an additional therapy. The methods and compositions, including combination therapies, enhance the therapeutic or protective effect, and/or increase the therapeutic effect of another therapy. Therapeutic and prophylactic methods and compositions can be provided in a combined amount effective to achieve the desired effect.

A LIPU therapy and/or APCs modified to express a chemokine (e.g., CXCL10) may be administered before, during, after, or in various combinations relative to an additional treatment. The administrations may be in intervals ranging from concurrently to minutes to days to weeks. In embodiments where a LIPU therapy and/or antigen presenting cells modified to express a chemokine (e.g., CXCL10) is provided to a patient separately from an additional agent, one would generally ensure that a significant period of time did not expire between the time of each delivery, such that the treatments would still be able to exert an advantageously combined effect on the patient. In such instances, it is contemplated that one may provide a patient with the first therapy and the second therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

The methods and compositions disclosed herein, such as a LIPU in combination with an antigen-presenting cell (e.g., modified to express CXCL10) may be further combined with a radiation therapy to treat a cancer (e.g., a brain cancer). Radiation therapies may trigger or promote interactions between dendritic cells and T cells in the glioma microenvironment (Ott et al., 2020). In preclinical models of glioma, the combination of whole brain radiation (WBRT) and signal transducer and activator of transcription 3 (STAT3) inhibition may triggers CD103+ dendritic cell and T cell interactions in the glioma microenvironment including antigen presentation and T cell activation. Without wishing to be bound by any theory, the radiation therapy may help or promote immune destruction of the cancer.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below the LIPU therapy and/or antigen presenting cells modified to express a chemokine (e.g., CXCL10) is “A” and the additional therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

A. Chemotherapy

A wide variety of chemotherapeutic agents may be used in accordance with the present embodiments. The term “chemotherapy” refers to the use of drugs to treat cancer. For example, temozolomide may be administered to the patient to treat a brain tumor (e.g., a glioblastoma). Temozolamide can administered (e.g., orally or intravenously) to the patient after a surgery to remove a brain tumor. In some embodiments, the chemotherapy is cisplatin, cyclophosphamide, etoposide, irinotecan, temozolamide, procarbazine, carmustine (BCNU), lomustine (CCNU), vincristine, a combination of drugs called PCV (i.e., a combination of lomustine, procarbazine, and vincristine), or a combination of the foregoing.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that immunotherapies may be used in combination with methods of the embodiments. In some embodiments, the immunotherapy is a checkpoint inhibitor, a monoclonal antibody, a cancer vaccine, an oncolytic virus, an adoptive T-cell therapy (ACT), or an adjuvant immunotherapy. The immunotherapy may be administered in combination with ultrasound disruption of the BBB and/or may be directly administered across the BBB (e.g., via intracranial injection, etc.).

In the context of cancer treatment, immunotherapeutics, generally, rely on the use of immune effector cells and molecules to target and destroy cancer cells. The immune effector may be, for example, a monoclonal antibody specific for some marker on the surface of a tumor cell. The monoclonal antibody can be, e.g., trastuzumab (Herceptin®), rituximab (Rituxan®), bevacizumab (Avastin®), nivolumab, ipilimumab, or pembrolizumab. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. In some embodiments, the immunotherapy includes depositing a cell (e.g., APC) in the tumor microenvironment. Without wishing to be bound by any theory, it is anticipated that the deposited cell may induce the migration of the effector cell(s) to the site and/or trigger immune cell activation.

In one aspect of immunotherapy, the tumor cell must bear some marker that is amenable to targeting, i.e., is not present on the majority of other healthy cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169); cytokine therapy, e.g., interferons α, β, and γ, IL-1, GM-CSF, and TNF; gene therapy, e.g., TNF, IL-1, IL-2, and p53 (U.S. Pat. Nos. 5,830,880 and 5,846,945); and monoclonal antibodies, e.g., anti-CD20, anti-ganglioside GM2, and anti-p185 (U.S. Pat. No. 5,824,311). It is contemplated that one or more anti-cancer therapies may be employed with the antibody therapies described herein.

Immunomodulatory agents include immune checkpoint inhibitors, agonists of co-stimulatory molecules, and antagonists of immune inhibitory molecules. The immunomodulatory agents may be drugs, such as small molecules, recombinant forms of ligand or receptors, or antibodies, such as human antibodies (e.g., International Patent Publication WO2015/016718; Pardoll 2012; both incorporated herein by reference). Known inhibitors of immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

Co-stimulatory molecules are ligands that interact with receptors on the surface of the immune cells, e.g., CD28, 4-1BB, OX40 (also known as CD134), ICOS, and GITR. As an example, the complete protein sequence of human OX40 has Genbank accession number NP_003318. In some embodiments, the immunomodulatory agent is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another co-stimulatory molecule that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. If desired, an anti-ICOS antibody can be used. Exemplary anti-ICOS antibodies include JTX-2011 (see, e.g., WO 2016/154177, WO 2018/187191) and GSK3359609 (see, e.g., WO 2016/059602).

Yet another co-stimulatory molecule that can be targeted in the methods provided herein is glucocorticoid-induced tumour necrosis factor receptor-related protein (GITR), also known as TNFRSF18 and AITR. The complete protein sequence of human GITR has Genbank accession number NP_004186. In some embodiments, the immunomodulatory agent is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, HL A-DRB1, HLA-DQA1, HLA-E, killer-cell immunoglobulin (KIR), lymphocyte activation gene-3 (LAG-3, also known as CD223), Mer tyrosine kinase (MerTK), NKG7, programmed death 1 (PD-1), programmed death-ligand 1 (PD-L1, also known as CD274), PDCD1LG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, immune checkpoint inhibitors targeting the PD-1 axis and/or CTLA-4 have received FDA approval broadly across diverse cancer types.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Pubin. Nos. WO 01/14424, WO 98/42752, WO 00/37504 (CP675,206, also known as tremelimumab; formerly ticilimumab): U.S. Pat. No. 6,207,156; Hurwitz et al., 1998; Camacho et al., 2004; and Mokyr et al., 1998 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Other immune inhibitory molecules that can be targeted for immunomodulation include STAT3 and indoleamine 2,3-dioxygenase (IDO). By way of example, the complete protein sequence of human IDO has Genbank accession number NP_002155. In some embodiments, the immunomodulatory agent is a small molecule IDO inhibitor. Exemplary small molecules include BMS-986205, epacadostat (INCB24360), and navoximod (GDC-0919).

In some embodiment, the immune therapy could be adoptive immunotherapy, which involves the transfer of autologous antigen-specific T-cells generated ex vivo. The T-cells used for adoptive immunotherapy can be generated either by expansion of antigen-specific T-cells or redirection of T-cells through genetic engineering (Park, Rosenberg el al. 2011). Isolation and transfer of tumor specific T-cells has been shown to be successful in treating melanoma. Novel specificities in T-cells have been successfully generated through the genetic transfer of transgenic T-cell receptors or chimeric antigen receptors (CARs) (Jena, Dotti el al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T-cells to be redirected against antigens expressed at the surface of tumor cells from various malignancies including lymphomas and solid tumors (Jena, Dotti el al. 2010).

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### VI. KITS

Certain aspects of the present invention may provide kits, such as therapeutic kits. For example, a kit may comprise one or more pharmaceutical composition as described herein and optionally instructions for their use. Kits may also comprise one or more devices for accomplishing administration of such compositions. For example, a subject kit may comprise a pharmaceutical composition and catheter for accomplishing direct intravenous injection of the composition.

Kits may comprise a container with a label. Suitable containers include, for example, bottles, vials, and test tubes. The containers may be formed from a variety of materials, such as glass or plastic. The container may hold a composition that includes a genetically engineered APCs that are effective for therapeutic or non-therapeutic applications, such as described above. The label on the container may indicate that the composition is used for a specific therapy or non-therapeutic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials & Methods

Cell culture. The murine GL261 and human U87 glioma cell lines were purchased from the National Cancer Institute and the National Institutes of Health, respectively. U87 cells were transfected to express EGFRvIII. This cellular product, EGFRvIII-U87 cells were a gift from Dr. Oliver Bogler (MD Anderson). Cell lines were maintained in Dulbecco's modified Eagle's medium (Life Technologies; Grand Island, NY) supplemented with 10% FBS (GIBCO), 1% penicillin/streptomycin, at 37° C. in a humidified atmosphere of 5% CO₂ and 95% air. Cells were passaged by trypsinizing for 3 minutes at 37° C., followed by neutralizing with medium containing FBS at 1:5 dilution.

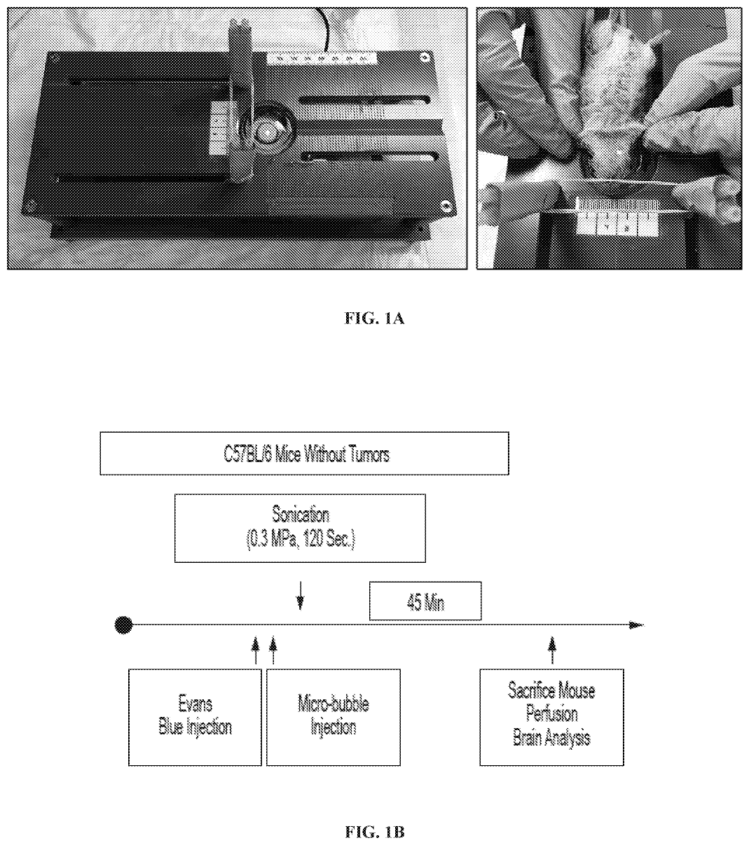

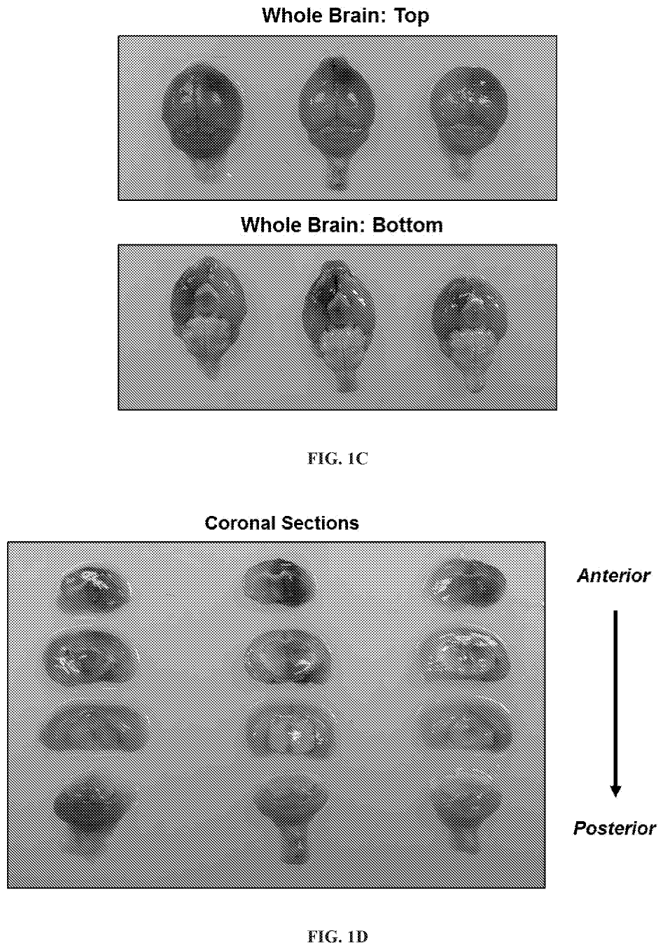

LIPU preclinical platform. The LIPU preclinical platform, designed by CarThera® (France), was used for all sonications. The setup corresponded to a 1.05-MHz frequency transducer placed in a tank of degassed water that ensured acoustic coupling (FIG. 1A). The transducer was operated at a center frequency of 1.05-MHz, a pulse repetition frequency of 1 Hz, a pulse length of 25,000 cycles (2.5% duty cycle, 23.2 msec total burst duration) and an in situ acoustic pressure level of 0.3 MPa. These parameters correspond to safe parameters determined in previous experiments in mice (Drean et al., 2019).

Ultrasound-induced BBBD procedures. For all US-induced BBBD procedures, hair was removed from the head with a clipper and hair removal cream (NAIR™) on the day prior to first treatment. Sonications were performed under general anesthesia after intraperitoneal injection of 150-200 μl of a mixture of Xylazine (10 mg/kg, AnaSed) and Ketamine HCl (100 mg/kg, Henry Schein). The feasibility and reproducibility of US-induced BBBD was assessed with the diffusion of Evans blue dye (Beccaria et al., 2013), which binds to albumin and does not freely cross the intact BBB (Uvama et al., 1988). The dye (Evans blue, Sigma-Aldrich) was diluted in saline, and 100 μl of the final solution, corresponding to a concentration of 100 mg/kg (Wang et al., 2009), was injected i.v. just before the i.v. injection of 200 μL of the ultrasound contrast agent (LUMASON®, Bracco Diagnostics). Mice were then transferred to the LIPU preclinical platform and the sonication was delivered for a total of two minutes. Forty-five minutes after sonication, mice were sacrificed after intracardiac perfusion of PBS. Brain was immediately removed and studied for Evans blue coloration. For the trafficking and treatment experiments, the immune therapeutic (anti-PD-1, EGFRvIII CAR T cells or APCs) was delivered i.v. just before i.v. injection of 200 μL of the ultrasound contrast agent (LUMASON®, Bracco Diagnostics). Mice were then transferred to the LIPU preclinical platform and the sonication was delivered for a total of two minutes. I.v. injections of Evans blue dye, ultrasound contrast agent and treatments were performed through the tail vein.

In vivo murine tumor models. All animal experiments were conducted in compliance with the guidelines for animal care and use established by the federal government and The University of Texas MD Anderson Cancer Center (MD Anderson) under protocol 00001544-RN01. To induce intracerebral tumors in C57BL/6 or NOD.Cg-Prkdcscid1L2Rγtm1Wjl/Sz (NSG, Jackson Laboratory) mice, GL261 or EGFRvIII-U87 cells were collected in logarithmic growth phase, loaded into a 50 μL syringe (Hamilton, Reno, NV) and injected 2 mm to the right of bregma and 4 mm below the surface of the skull at the coronal suture using a stereotactic frame (Stoelting, Wood Dale, IL). The intracranial tumorigenic dose for GL261 cells and EGFRvIII-U87 cells were 5×10⁴ and 1.5×10⁵ in a total volume of 2 μL and 4 μL, respectively. Mice were randomly assigned to control or treatment groups after tumor implantation. The animals were observed daily, and when they showed signs of neurological deficit (lethargy, failure to ambulate, lack of feeding, or loss of >20% body weight), they were compassionately killed. These symptoms typically occurred within 48 hours before death. Brains were collected after cardiac perfusion with PBS, placed in 10% formalin and embedded in paraffin.

Ani-PD-1 fluorescent tagging and in vivo biodistribution analysis. The anti-PD-1 antibody was fluorescently tagged with Alexa fluor 647 using the SAIVI Rapid Ab Labeling Kit according to the manufacturer's recommendations (S30044, Invitrogen, CA). Briefly, 1 mg of the antibody at a concentration of 2 mg/mL was incubated with the Alexa 647 dye for one hour at room temperature with gentle stirring. A 3-cm column was prepared by using the resin in the kit and washed twice with elution buffer before loading the sample. The labeled antibody was loaded onto the column, and all the eluted fractions were collected. The first-eluted colored bands contained the labeled antibody. Absorbance of the purified conjugated antibody was measured at both A280 and 650 nm and the protein concentrations were calculated using a Nanodrop 1000 spectrophotometer (Thermo Scientific, CA).

Treatment with PD-1 monoclonal antibodies. Monoclonal antibodies against PD-1 (BE0146, clone RMP1-14) were obtained from BioXCell (New Hampshire, USA). After tumor implantation, mice were randomized into groups with an equal balance between groups of female and male animals. Mice were treated with i.v. injection of either anti-PD-1 antibody (200 μg/mouse; Bio X Cell) alone, anti-PD-1 antibody without ultrasound, or isotype control IgG (200 μg/mouse; Bio X Cell) alone. Treatments were performed on days 3, 7, 10, 14, and 17 after i.c. implantation. Animals that died from tumor progression prior to the initiation of at least three treatments were not included in survival analysis. Mice were euthanized when they exhibited neurological morbidity. The brains were collected and fixed in 10% formalin and paraffin embedded for histological analysis.

EGFRvIII (AR construction. The CAR T cells were engineered using an EGFRvIII CAR made by fusing the scFv of the EGFRvIII-specific monoclonal antibody mAB 139. The scFv was attached to an IgG4 stalk, and intracellular CD28 and CD3-zeta signaling domains as previously described (Caruso et al., 2019). A shortened ex vivo produced strategy was used to create the CAR T cells with an improved immune phenotype associated with greater therapeutic outcomes (Caruso et al., 2019). The CAR plasmid was modified to express a firefly luciferase (ffLuc) fluorescent reporter for CAR monitoring in vivo. Human healthy donor PBMCs underwent negative selection for CD3+ T cells followed by electroporation of the SB transposon containing CAR with SB transposase SB11. CAR T cells were expanded in vitro over 14 days by combining them with irradiated activating and propagating cells (AaPCs) in addition to IL-2 and IL-21. Prior to murine CAR administration, CAR expression was quantified with flow cytometry with an anti-Fc antibody to detect the IgG4 portion of the CAR. 1.5×10⁷ EGFRvIII/ffLuc CAR T cells were administered i.v. for the BLI experiments and for treatment.

CXCL9 and CXCL10 gene transduction into F4/80+ antigen presenting cells. To transduce the CXCL9 and CXCL10 immune chemokine genes into APCs, lentivirus that encoded either the cDNA of murine CXCL9 or CXCL10 was prepared. Both CXCL9 (Cat #MR200667L3) and CXCL10 (Cat #MR200291L4) gene transfer plasmids were purchased from Origene (Rockville, MD). Mouse bone marrow cells were extracted under sterile conditions from 6-week old female C57BL/6 mice as previously described (Heimberger et al., 2000). Red blood cells were lysed with 0.84% ammonium chloride and then washed with PBS. Lymphocytes, granulocytes, and Ia+ cells were depleted using complement lysis using low-tox rabbit complement (Cedarlane) with the following monoclonal antibodies (MAb). GK1.5 (CD4), 2.43 (CD8), RA3-3A1/6.1 (CD45R), B21-2 (Class II) and RB6-8C5 (Gr-1). Depleted cells were plated in six-well cell culture plates (3×10⁶ cells/well) in RPMI medium 1640 supplemented with 5% (vol/vol) fetal bovine serum, 50 mM β-mercaptoethanol, 10 mM Hepes, 2 mM glutamine, 100 U/mL penicillin, 100 mg/mL streptomycin, and 1000 U/mL of GM-CSF (Immunex, Seattle, WA) or in AIM-V (Gibco BRL) supplemented with 1000 U/mL of GM-CSF. Floating cells were removed on day 3 with replacement of fresh medium containing 1000 U/mL GM-CSF. On day 7 nonadherent cells were collected and re-plated in 100-mm Petri dishes (1-10⁷ cells/plate). On day 10, non-adherent cells were removed for phenotypic analysis via FACscan to analyze the surface markers expression of F4/80 and CD11c (Sheng et al., 2017). The cells were infected with 5 mL of freshly prepared lentivirus (TU=6.5×10⁶/mL), encoding either CXCL9 or CXCL10. Chemokine production was measured by ELISA (Cat #ab203364 and ab214563, Abcam, Cambridge, MD) according to the manufacturer's instructions.

### Example 2—LIPU Reproducibly Opens the Blood-Brain Barrier in Pre-Clinical Models

The pre-clinical platform used for opening the BBB in murine glioblastoma models consists of an ultrasound transducer surrounded by a compartment of degassed water with a laser aligned on the center of the transducer. To enhance reproducibility of the sonicated areas, the shaved mouse head is placed in contact with the surface of the degassed water and the area of tumor implantation is aligned with the laser dot, which indicates the center of the transducer. Head stabilization allows for motionless sonication to the CNS (FIG. 1A). C57BL/6 mice without tumors were administered i.v. Evans blue followed immediately by ultrasound and 45 minutes time for the dye to circulate. Mice were then perfused and brains were photographed following removal (FIG. 1B). Images of the superior and inferior whole brains show Evans blue penetration in the anterolateral brain region targeted by the ultrasound beam (FIG. 1C). Whole brains were then sectioned coronally and photographed to investigate the presence of BBBD in the depth of the brain parenchyma. Coronal sections show Evans blue distribution throughout the depth of the brain from superior to inferior, in the anterior part of the hemisphere (FIG. 1D). This confirms the feasibility of reproducible and targeted BBBD in regions of potential tumor implantation.

### Example 3—Anti-PD-1 CNS Delivery is Enhanced with LIPU

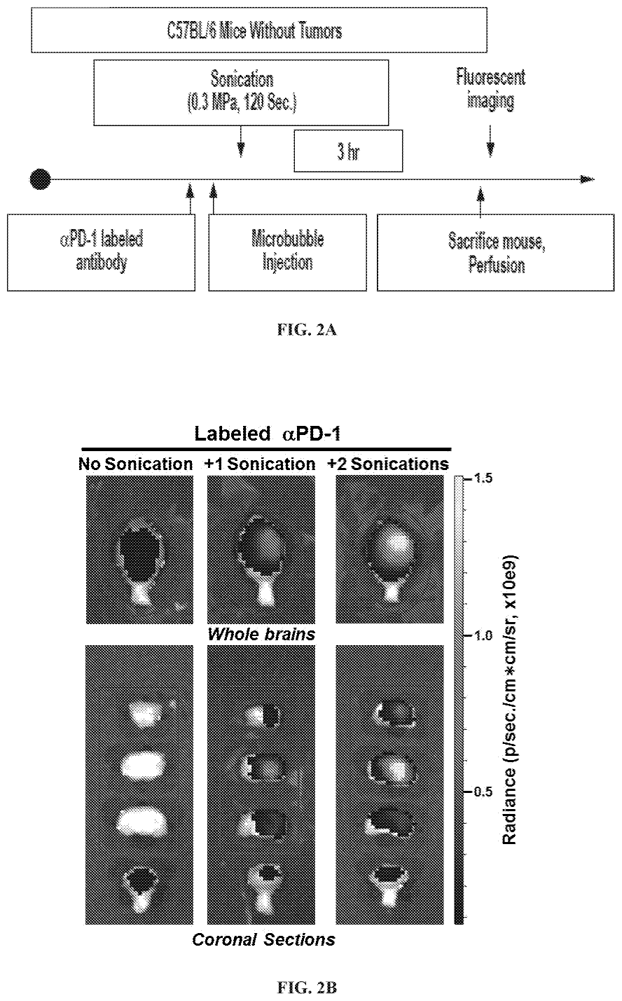

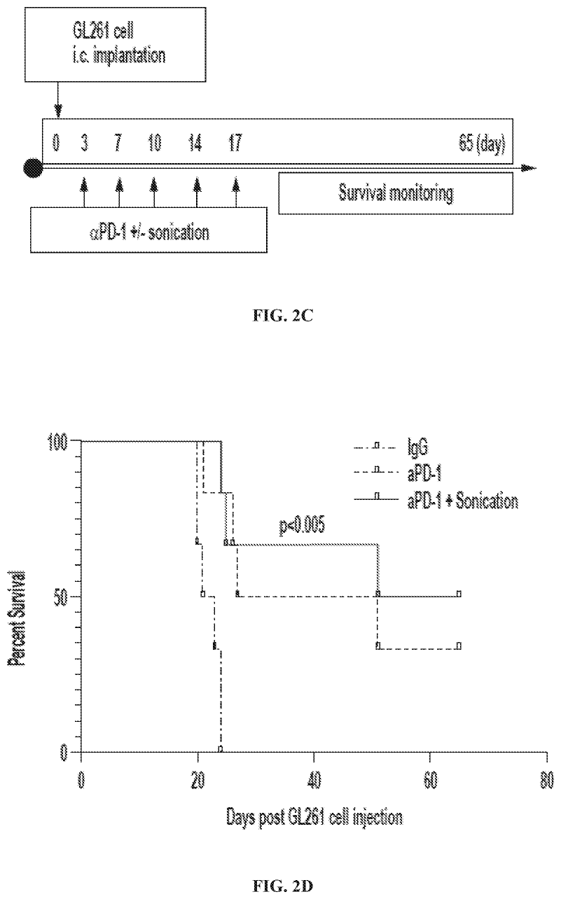

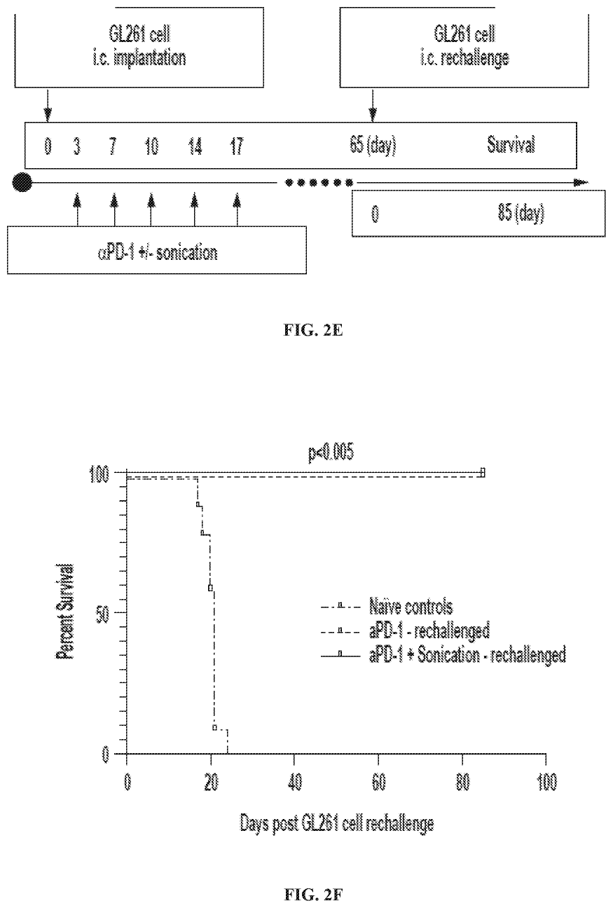

Anti-PD-1 was administered in combination with ultrasound BBBD to determine if antibodies limited by the BBB could be better delivered and provide improved function for glioblastoma treatment. Anti-PD-1 was fluorescently tagged with Alexa fluor 647 to allow for determination of antibody delivery and relative concentration in murine brains. Labeled anti-PD-1 was administered i.v. with ultrasound followed by a period of 3 hours for antibody circulation. Fluorescent imaging was done on the brains using the IVIS 200 fluorescence imager immediately following mouse perfusion and brain dissection (FIG. 2A). Anti-PD-1 given alone showed low levels of antibody infiltration in the cerebellum with minimal focal enhancement of delivery over the potential site of tumor implantation. With LIPU, there was enhanced and localized labeled anti-PD-1 delivery to the anterolateral brain region, which was further enhanced with additional sonication treatments (FIG. 2B).

### Example 4—LIPU Further Potentiates the Impact of Anti-PD-1 Against CNS Gliomas

Since the anti-PD-1 appeared to be better delivered in combination with ultrasound, it was evaluated whether or not this improvement would have an effect in glioma-bearing mice. Mice bearing established intracranial (i.c.) GL261 were treated twice a week for 2.5 weeks i.v. with either IgG, anti-PD-1, or anti-PD-1 in combination with ultrasound (FIG. 2C). There was an increase in the median survival (MS) to 58 days in mice treated with the combination of anti-PD-1 and BBB opening ultrasound relative to both the IgG group (MS: 22 days; p<0.005) and the anti-PD-1 group (MS: 39 days; p=0.6226) (FIG. 2D). At 65 days after the initial (i.c.) implantation of GL261, 5 long-term survivors remained (anti-PD-1, n=2; anti-PD-1+US, n=3). These mice, along with age matched naive controls (n=10) were re-challenged in the contralateral hemisphere to emulate a tumor recurrence (FIG. 2E). All long-term survivors treated with anti-PD-1 or anti-PD-1 with ultrasound remained alive following re-challenge while all age matched controls died with a MS of 21 days (p=0.0148, p<0.005, respectively), suggesting an immunologically protective effect (FIG. 2F). Multiple treatments with anti-PD-1 and ultrasound were found to be safe as there were no findings of adverse events, behavior changes, or signs of neurologic toxicity in mice. Repeated dual i.v. treatments did lead to loss of i.v. access in some mice and rarely tail necrosis leading to partial tail amputation. This limited the number of possible treatment sessions and may impact future experimental designs using schedules beyond 5 treatments.

### Example 5—LIPU Enhances CAR T Cells Trafficking to the Glioma Microenvironment

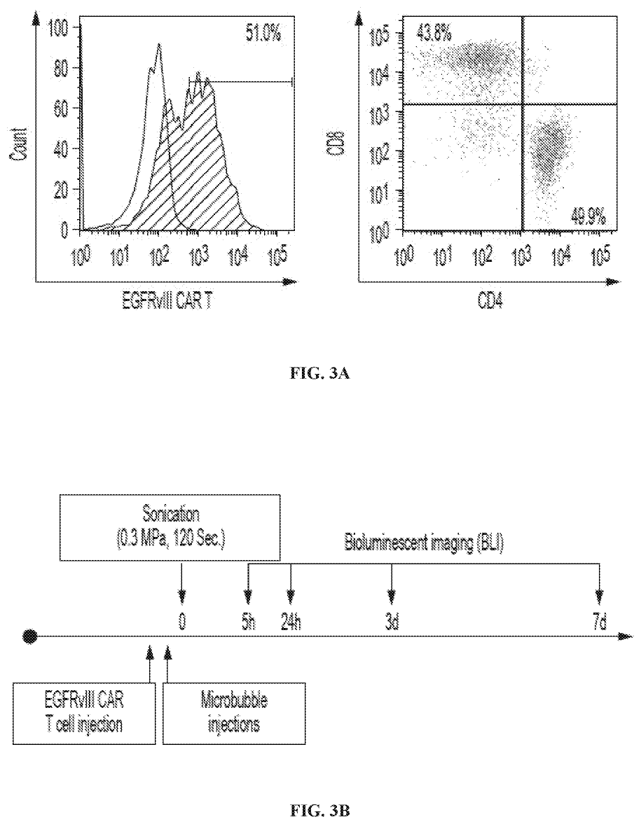

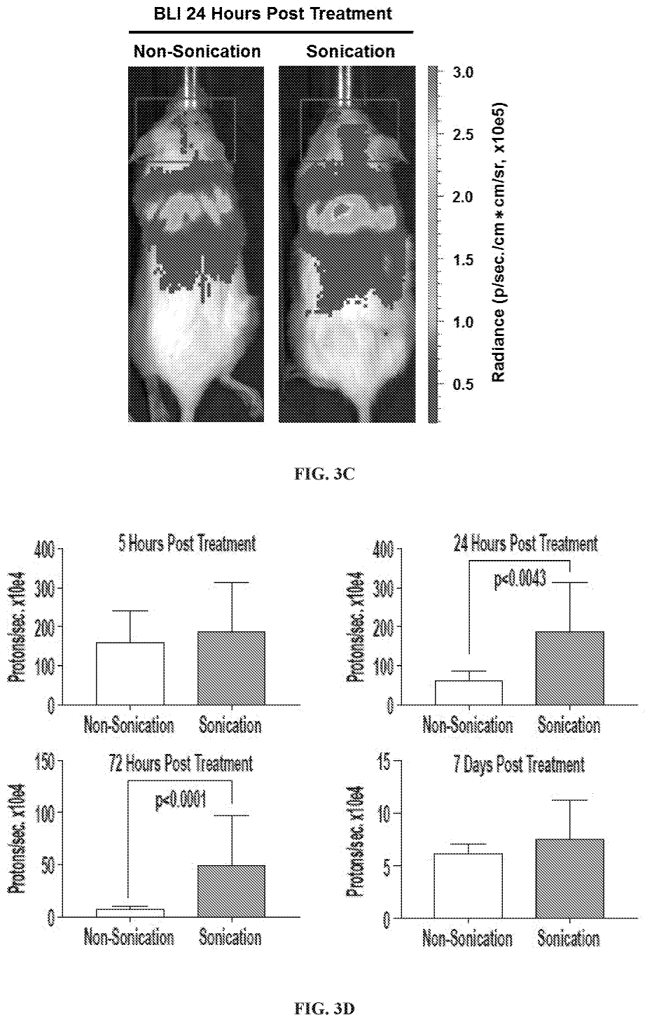

To determine if cellular therapies, such as CAR T cells, could be improved by increasing the distribution of cells to the tumor microenvironment with LIPU, the delivery and persistence of CAR T cells administered with and without BBB opening were directly compared. The CAR plasmid was modified to express a firefly luciferase (ffLuc) fluorescent reporter for CAR monitoring in vivo. Prior to infusion, the expression of the CAR was verified, and that the T cell population contained both CD4 and CD8 T cells (FIG. 3A), which have been shown to generate a superior response (Sommermeyer et al., 2016; Turtle et al., 2016). FfLuc labeled EGFRvIII CAR T cells were used to determine if ultrasound mediated BBBD can affect cellular therapy trafficking and persistence in vivo (FIG. 3B). BLI demonstrated that most CARs were distributed to the liver and lungs making up the increased thoracic signal with a small fraction distributing to the brain (FIG. 3C). In mice treated with both the EGFRvIII CAR T cells and BBB opening, starting 24 hours after treatment (p<0.005) and extending to 72 hours (p<0.0001), there was a significant increase in CAR T bioluminescence (FIG. 3D) indicating LIPU enhances the focal delivery of the CAR to the brain.

### Example 6—Ultrasound Administration of EGFRVIII-CAR T Cells Increases Survival in Murine Glioblastoma Model

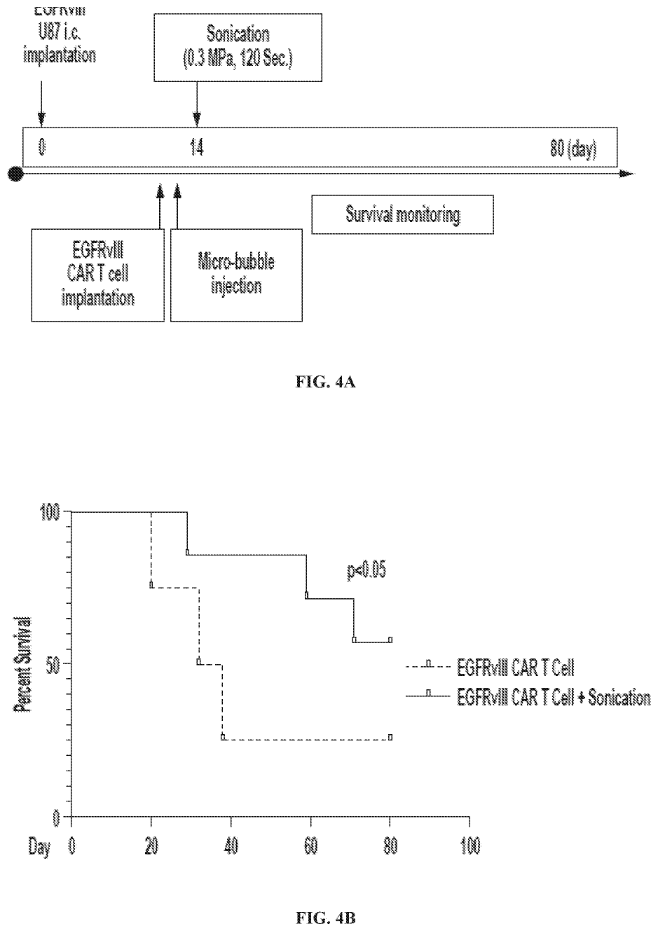

Since CAR T cells administered in combination with ultrasound were found to have increased trafficking to the tumor and a longer persistence, it was investigated whether this difference could lead to a potential biologically significant effect in vivo. NSG mice were implanted with EGFRvIII expressing U87 tumors. Mice treated with EGFRvIII/ffLuc CAR T cells i.v. with ultrasound 14 days after tumor implantation (FIG. 4A) were found to have an undefined median survival relative to mice treated with CAR T cells alone that had a MS of 35 days (p<0.05) (FIG. 4B). Administration of EGFRvIII CAR T cells in both groups did show mild signs of systemic toxicity observed by hunched posturing for up to 3 days following treatment. However, mice returned to baseline without long-term adverse effects and did not demonstrate any signs of neurologic toxicity.

### Example 7—Antigen Presenting Cells (APCs) Engineered to Produce the T Cell CXCL10 Chemokine Show Therapeutic Efficacy with LIPU

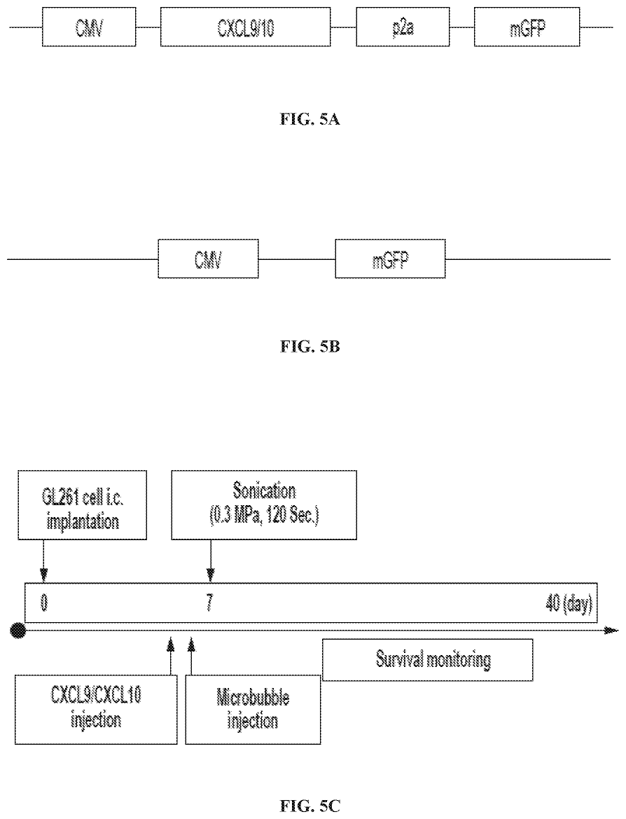

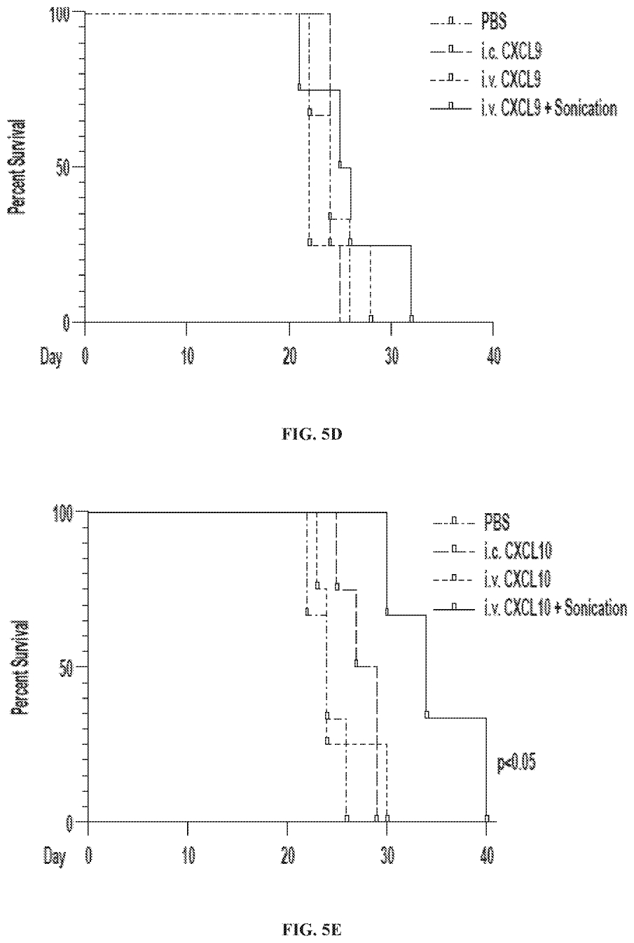

Studies using CAR T cells demonstrate that ultrasound can be used as a method for enhancing the administration of cellular immunotherapies. To extend these findings, and based on the paucity of APCs within the tumor microenvironment (Wang et al., 2017a), it was hypothesized that an APC could be modified to express a T cell chemokine that could enhance localized immune activation and that US-induced BBBD would enhance delivery at the site of tumor antigens. Murine APCs were isolated from the bone marrow of C57BL/6 mice and modified by lentivirus transduction to encode the cDNA of CXCL9 or CXCL10 (van der Woude et al., 2017) (FIG. 5A) and mGFP for cell tagging (FIG. 5B). APCs underwent phenotypic analysis by flow cytometry for surface markers of F4/80 and CD11c, and chemokine production of CXCL9 or CXCL10 by ELISA test in the range of 3,392 pg/mL and 2987 pg/mL, respectively, which showed stable transduction even after storage at −80° C. To ascertain if there was a therapeutic effect, C57BL/6 mice were implanted with GL261 cells and treated with 1×10⁶ CXCL9 or CXCL10 macrophages given i.c., or i.v. with or without ultrasound (FIG. 5C). Mice tolerated treatments well without any adverse events, behavior changes, or neurologic toxicity. Survival analysis of mice treated with CXCL9 macrophages using three different treatment administration methods, in comparison to the PBS control, showed no significant benefit in murine survival (FIG. 5D). However, mice treated with CXCL10 expressing APCs showed a significant increase in survival when administered i.c. compared to PBS, demonstrating a therapeutic effect when the cellular therapy is administered directly to the tumor microenvironment (p<0.05). When the same concentration of CXCL10 APCs were administered intravenously, there was no benefit on murine survival when compared to the PBS group (p=0.6041). When this cellular therapy was given i.v. in combination with US-induced BBBD, there was a significant increase in survival relative to PBS (p<0.05), intravenous only (p<0.05), and i.c. (p<0.05) groups (FIG. 5E).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Patent Application Publication No. 2011/0008369
U.S. Patent Application Publication No. 2014/022021
U.S. Patent Application Publication No. 2014/0294898
U.S. Pat. No. 5,801,005
U.S. Pat. No. 5,739,169
U.S. Pat. No. 5,830,880
U.S. Pat. No. 5,846,945
U.S. Pat. No. 5,824,311
U.S. Pat. No. 8,119,129
U.S. Pat. No. 6,207,156
U.S. Pat. No. 8,017,114
U.S. Pat. No. 8,329,867
U.S. Pat. No. 7,829,673
U.S. Pat. No. 5,844,905
U.S. Pat. No. 5,885,796
U.S. Pat. No. 5,760,395
U.S. Pat. No. 4,870,287
U.S. Pat. No. 8,735,553
U.S. Pat. No. 8,354,509
U.S. Pat. No. 8,008,449
WO 1995001994
WO 98/42752
WO 1998042752
WO 00/37504
WO 2001/014424
WO 01/14424
WO 2000/037504
WO 2006/105021
WO 2009/101611
WO 2009/114335
WO 2010/027827
WO 2011/066342
WO 2015/016718
WO 2015/033299
WO 2015/033301
WO 2015/097536
WO 2015/116539
WO 2016/028656
WO 2016/059602
WO 2016/154177
WO 2016/207717
WO 2017/019846
WO 2017/030823
WO 2017/130076
WO 2017/137830
WO 2017/175058
WO 2017/182672
WO 2017/220569
WO 2018/002339
WO 2018/091740
WO 2018/187191
WO 2018/201096
WO 2018/202649
Alkins et al., Cancer Res.: 73(6):1892-1899, 2013.
Belcaid et al., PLoS One; 9(7):e101764, 2014.
Camacho el al., J Clin Oncology, 22(145): Abstract No. 2505 (antibody CP-675206), 2004.
Carpentier et al., Sci Transl Med.; 8(343):343re342, 2016.
Caruso et al., J Neurooncol.; 145(3):429-439, 2019.
Chai et al., J. Control. Release 192, 1-9, 2014.
Chongsathidkiet et al., Nat Med.; 24(9):1459-1468, 2018.
Drean et al., J Neurooncol.; 144(1):33-41, 2019.
Heimberger et al., J Neuroimmunol.; 103(1):16-25, 2000.
Hurwitz el al., Proc Natl Acad Sci USA, 95(17): 10067-10071, 1998.
Hussain et al., Neuro Oncol.; 8(3):261-279, 2006.
Hynynen et al., Noninvasive MR imaging-guided focal opening of the blood-brain barrier in rabbits. Radiology; 220(3):640-646, 2001.
Hynynen et al., Neuroimage 24, 12-20, 2005.
Hynynen et al., Acta Neurochir. Suppl. 86, 555-558, 2003.
Jena, Dotti et al. 2010.
Kinoshita et al., Proc Natl Acad Sci USA.; 103(31):11719-11723, 2006.
Ledford, Nature News; doi:10.1038/news.2011.319, 2011.
Louveau et al., Nature; 523(7560):337-341, 2015.
Maiuolo et al., Int. J. Mol. Sci. 19:2693, 2018.
Mitchell et al., Nature; 519(7543):366-369, 2015.
Mokyr et al., Cancer Res, 58:5301-5304, 1998.
Nefedova et al., J Immunol.; 175(7):4338-4346, 2005.
Nelson et al., Biochim. Biophys. Acta 1862, 887-900, 2016.
Nwajei et al., Neuro-Oncology; Vol. 17, Issue suppl. 5, November 2015.
Ott et al., Clin Cancer Res.; 26(18):4983-4994, September 2020.
Ott et al., Immunological reprogramming in the CNS tumor environment and therapeutic efficacy of radiotherapy with stat3 blockade. Paper presented at: SNO2019.
Pardoll, Nat Rev Cancer, 12(4): 252-264, 2012.
Park et al., J. Control. Release 162, 134-142, 2012.
Park, Rosenberg et al. 2011.
Reardon et al., Cancer Immunol Res.; 4(2):124-135, 2016.
Reuben et al., Cancer Discov.; 7(10):1088-1097, 2017.
Ribas et al., Cancer Immunol Res.; 4(3):194-203, 2016.
Sabins et al., Front Immunol.; 7:221, 2016.
Schoknecht et al., Semin. Cell Dev. Biol. 38, 35-42.
Sheng el al., Cell Rep.; 21(5):1203-1214, 2017.
Sommermeyer et al., Leukemia; 30(2):492-500, 2016.
Song et al., Theranostics; 7(1): 144-152, 2017.
Sweeney et al., Nat. Neurosci. 21, 1318-1331, 2018.
Turtle et al., J Clin Invest.; 126(6):2123-2138, 2016.
Uvama e al., 1988.
van der Woude et al., 2017
Vom Berg et al., J Exp Med.; 210(13):2803-2811, 2013
Wang et al., Cancer Cell; 32(1):42-56 e46, 2017a.
Wang et al., Cancer Immunol Res.; 5(2):148-156, 2017b.
Wang el al., Cancer Cell; 33(1):152, 2018.
Wang et al., J Ultrasound Med.; 28(11):1501-1509, 2009.
Wei el al., J Clin Invest.; 129(1):137-149, 2019.
Wherry, Nat Immunol.; 12(6):492-499, 2011.
Woroniecka et al., Clin Cancer Res.; 24(17):4175-4186, 2018.
Zhao el al., Cell 163, 1064-1078, 2015.

What is claimed is:

1. A method of treating a brain cancer in a patient in need thereof, the method comprising administering to the patient an effective amount of antigen presenting cells (APCs) in conjunction with ultrasound blood brain barrier disruption, wherein the antigen presenting cells have been genetically modified to express a chemokine.

2. The method of claim 1, wherein the chemokine is CXCL9 or CXCL10.

3. The method of claim 2, wherein the chemokine is CXCL10.

4. The method of any one of claims 1-3, wherein the administration is intravenous.

5. The method of claim 1 or 4, wherein the ultrasound blood brain barrier disruption is a low-intensity pulsed ultrasound (LIPU) therapy.

6. The method of any one of claims 1-5, wherein the patient has previously failed to respond to an immunotherapy.

7. The method of any one of claims 1-6, wherein the APC is genetically modified to reduce or prevent immune suppression by the subject.

8. The APC of any one of claims 1-7, wherein the APC is genetically modified to increase expression of MHC.

9. The APC of any one of claims 1-8, wherein the APC is genetically modified to increase expression of one or more co-stimulatory molecules.

10. The method of any one of claims 1-9, wherein the APCs are derived from autologous cells from patient.

11. The method of any one of claims 1-10, wherein the APCs are derived from allogeneic cells.

12. The method of any one of claims 1-11, wherein the APCs are professional APCs, dendritic cells (DCs), macrophages, or B cells.

13. The method of any one of claims 1-12, wherein the method further comprises administering an immune checkpoint inhibitor to the subject.

14. The method of claim 13, wherein the immune checkpoint inhibitor comprises an anti-PD1 antibody.

15. The method of claim 14, wherein the anti-PD1 antibody comprises nivolumab, pembrolizumab, pidilizumab, AMP-223, AMP-514, cemiplimab, or PDR-001.

16. The method of any one of claims 1-15, wherein the brain cancer is a glioma, a glioblastoma, a glioblastoma multiforme, an astrocytoma, a pituitary adenoma, an acoustic neuroma, a medulloblastoma, a meningioma, a haemangioblastoma, an ependymoma, or a subependymoma.

17. The method of any one of claims 1-16, further comprising administering a further anti-cancer therapy to the patient.

18. The method of claim 17, wherein the further anti-cancer therapy is a chemotherapy, an immunotherapy, a radiotherapy, a gene therapy, surgery, a hormonal therapy, an anti-angiogenic therapy, or a cytokine therapy.

19. An isolated, engineered antigen presenting cell (APC) that comprises a transgene for expressing at least one chemokine.

20. The APC of claim 19, wherein the chemokine is CXCL9 or CXCL10.

21. The APC of claim 19 or 20, wherein the chemokine is CXCL10.

22. The APC of any one of claims 19-21, wherein the APC is genetically modified to prevent its immune suppression.

23. The APC of any one of claims 19-22, wherein the APC is genetically modified to fortify expression of MHC.

24. The APC of any one of claims 19-23, wherein the APC is genetically modified to fortify expression of co-stimulatory molecules.

25. The APC of any one of claims 19-24, wherein the APC is a professional APC, a macrophage, a dendritic cell, a T cell, or a B cell.

26. A pharmaceutical composition comprising the APC of any one of claims 19-25 and at least one pharmaceutically acceptable carrier, diluent, or excipient.

27. The composition of claim 26, further comprising an immune checkpoint inhibitor.

28. The pharmaceutical composition of claim 26, for use in the treatment of a brain cancer.

29. A method of treating a brain cancer in a patient in need thereof, the method comprising administering to the patient an effective amount of the composition of claim 26.

30. The method of claim 29, wherein the administration is intracranial.

31. The method of claim 29, wherein the administration is intravenous.

32. The method of claim 31, wherein the patient is subjected to ultrasound blood brain barrier disruption.

33. The method of claim 32, wherein the blood brain barrier disruption is low-intensity pulsed ultrasound (LIPU) therapy.

34. The method of any one of claims 29-33, wherein the patient has previously failed to response to an immunotherapy.

35. The method of any one of claims 29-33, wherein the method prevents tumor recurrence.

36. The method of any one of claims 29-35, wherein the APCs are autologous to the patient.

37. The method of any one of claims 29-35, wherein the APCs are allogeneic to the patient.

38. The method of any one of claims 29-37, wherein the brain cancer is a glioma, a glioblastoma, a glioblastoma multiforme, an astrocytoma, a pituitary adenoma, an acoustic neuroma, a medulloblastoma, a meningioma, a haemangioblastoma, an ependymoma, or a subependymoma.

39. The method of any one of claims 29-38, further comprising administering a further therapy to the patient.

40. The method of claim 39, wherein the further therapy is a chemotherapy, an immunotherapy, a radiotherapy, a gene therapy, surgery, a hormonal therapy, an anti-angiogenic therapy, or a cytokine therapy.

41. The method of claim 40, wherein the immunotherapy comprises an immune checkpoint inhibitor.

42. The method of claim 41, wherein the immune checkpoint inhibitor comprises an anti-PD1 antibody.

43. The method of claim 42, wherein the anti-PD1 antibody comprises nivolumab, pembrolizumab, pidilizumab, AMP-223, AMP-514, cemiplimab, or PDR-001.

44. The method of claim 40, wherein the immunotherapy comprises an adoptive T-cell therapy.
